(19) United States
(12) Patent Application Publication
(10) Pub. No.: US 2023/0233797 A1
(43) Pub. Date: Jul. 27, 2023

(54) Catheter Brush Device

(71) Applicants: Kimberly Cooper, Odell (CA); Lori McPherson, Presque Isle, ME (US)

(72) Inventors: Kimberly Cooper, Odell (CA); Lori McPherson, Presque Isle, ME (US)

(21) Appl. No.: 18/047,826

(22) Filed: Oct. 19, 2022

Related U.S. Application Data
(60) Provisional application No. 63/302,259, filed on Jan. 24, 2022.

(51) Int. Cl. A61M 25/00

(52) U.S. Cl. CPC A61M 25/0017; A61M 25/002; A61M 2025/0019; A61M 2210/1085

(57) ABSTRACT

The present invention relates to a novel catheter brush device. The device is designed to offer a more effective method of removing blood clots and debris from the bladder without requiring additional surgery. The device comprises a brush with a blunt end with soft bristles, as well as a loop end that acts as a handle. Users remove the drainage tube from the catheter and insert the brush through the opening to the tip of the catheter. The brush is then moved in a gentle, circular motion to clear any clotting and blockages from the catheter. The brush is then removed, and the drainage tube reattached, such that urine can flow freely through the catheter. If sterility is maintained, process may be repeated as needed.

### CROSS-REFERENCE TO RELATED APPLICATION

The present application claims priority to, and the benefit of, U.S. Provisional Application No. 63/302,259, which was filed on Jan. 24, 2022, and is incorporated herein by reference in its entirety.

### FIELD OF THE INVENTION

The present invention relates generally to the field of catheter brush devices. More specifically, the present invention relates to an improved catheter brush device that provides users with a modified brush device capable of removing blood clots and blockages from a Foley catheter, especially during post-operative care. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in Foley catheters. A Foley catheter is a flexible tube that is usually passed through the urethra and into the bladder. The tube has two or more separate channels, or lumens, running down its length. One lumen is open at both ends and allows urine to drain out into a collection bag. Another lumen, or side lumen, typically has a valve on the outside end and connects to a balloon at the tip; the balloon is inflated with sterile water when it lies inside the bladder, in order to stop it from slipping out. Foley catheters are commonly made from silicone rubber or natural rubber. A temperature, or thermistor, sensor can be placed inside one of the Foley catheter’s side lumen with a sensing tip positioned at the catheter’s proximal end. The proximal end is usually beveled for atraumatic insertion. The temperature sensor is used to measure a patient’s bladder or core temperature.

Foley catheters can become clogged or blocked due to blood clots or other material within the bladder. The current practice of irrigating the clots back into the bladder is painful and often unsuccessful. Additional trauma to a patient is caused when the plugged catheter must be removed. Furthermore, the force on the catheter being pulled from a patient tends to stretch the elastic catheter material which can damage the catheter. If the catheter is damaged during removal, the pull on the catheter can make removal of the catheter more difficult and can cause urethral abrasion. Additionally, removing the catheter can cause more inflammation to the operative site, as a new sterile, often larger catheter will be placed to help pass the clots. Often, this catheter cannot be placed unless a urologist is called and a stylet is used, which allows for a more rigid and dangerous insertion of a catheter. If this is not successful, then a surgical procedure is performed to insert a suprapubic catheter. This is a surgical incision through the abdomen into the bladder to drain urine and prevent the bladder from rupturing. Several surgeries on a patient can lead to serious complications and further injuries.

Thus, it is necessary for users to have a more effective method of removing blood clots and other debris from the bladder without requiring additional surgery. Generally, the catheter brush device provides users with a modified brush capable of removing blood clots and blockages from a Foley catheter during post-operative care. Therefore, the device maintains optimal flow of urine through the catheter without unnecessary procedures.

Therefore, there exists a long-felt need in the art for a catheter brush device that provides users with a modified brush capable of removing blood clots and blockages from a Foley catheter during post-operative care. There is also a long-felt need in the art for a catheter brush device that features a blunt end brush with a loop end for holding, which allows users to insert the sterile brush into the catheter to secure and remove the clot. Further, there is a long-felt need in the art for a catheter brush device that allows the process of clot removal to be repeated, if sterility is maintained, to safely remove all clots without causing additional pain and trauma to a patient. Moreover, there is a long-felt need in the art for a device that maintains optimal flow for urine through the catheter when the drainage tubing is reconnected. Further, there is a long-felt need in the art for a catheter brush device that offers a more effective method of removing blood clots and debris from the bladder without requiring additional surgery. Finally, there is a long-felt need in the art for a catheter brush device that reduces trauma to a patient.

The subject matter disclosed and claimed herein, in one embodiment thereof, comprises a catheter brush device. The device is designed to offer a more effective method of removing blood clots and debris from the bladder without requiring additional surgery. The device comprises a brush with a blunt end with soft bristles, as well as a loop end that acts as a handle. Users remove the drainage tube from the catheter and insert the brush through the opening to the tip of the catheter. The brush is always contained in the catheter, so there is no risk of perforation of the bladder. The brush is then moved in a gentle, circular motion to clear any clotting and blockages from the catheter. The brush is then removed and the drainage tube reattached, such that urine can flow freely through the catheter. Thus, blood clots and blockages are removed without additional surgical incisions and reduced trauma to a patient.

In this manner, the catheter brush device of the present invention accomplishes all of the foregoing objectives and provides users with a device that removes blood clots and blockages from catheters without trauma to the patient. The device allows a user to maintain optimal urine flow through a catheter. The device saves time and effort without requiring additional surgeries.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a catheter brush device. The device is designed to offer a more effective method of removing blood clots and debris from the bladder without requiring additional surgery. The device comprises a brush with a blunt end with soft bristles, as well as a loop end that acts as a handle. Users remove the drainage tube from the catheter and insert the brush through the opening to the tip of the catheter. The brush is then moved in a gentle, circular motion to clear any clotting and blockages from the catheter. The brush is then removed, and the drainage tube reattached, such that urine can flow freely through the catheter.

The catheter brush device is typically used with a standard Foley catheter as is known in the art but can be utilized with any suitable catheter as is known in the art, depending on the needs and/or wants of a user. Generally, the Foley catheter comprises a proximal end and a distal end. The proximal end comprises side drainage openings, wherein as the distal end of the catheter preferably has three lumen endings. Typically, the largest diameter lumen ending is preferably the drainage lumen extending from the side drainage openings to the drainage lumen ending. This drainage lumen ending is preferably used to drain urine from the bladder. The drainage lumen ending preferably has a fitting to connect the lumen to a catheter bag (not shown). Furthermore, the catheter lumens are preferably made of silicon, or any other suitable flexible material as is known in the art.

In one embodiment, the catheter brush device comprises a brush component with a first end comprising a blunt end with a plurality of soft bristles. The blunt end can be any suitable shape as is known in the art, as long as the end does not comprise any sharp edges to damage the catheter during insertion. Typically, the blunt end is a rounded end. The plurality of soft bristles are then secured to the blunt end via any suitable securing means as is known in the art, such as glue, adhesive, etc. Any suitable number of bristles can be utilized as is known in the art, depending on the needs and/or wants of a user. Typically, bristles are secured all around the blunt end and down the sides forming a tube-like shape of bristles at the blunt end. In one embodiment, more bristles can be utilized on the catheter brush device for removing large blood clots and clogs within the catheter, and less bristles can be utilized on the catheter brush device for removing smaller blood clots and clogs within the catheter.

In another embodiment, the brush component of the catheter brush device comprises a second end comprising a wire loop that acts as a handle. Typically, the second end can comprise any suitable shape as is known in the art, such as a circle, square, rectangle, etc., as long as a user can easily grasp the second end like a handle when using the device. In one embodiment, the second end can be straight and not have a loop or other shape. In this embodiment, the user would merely grasp the straight second end to manipulate the device.

In one embodiment, the brush will always be “notched/marked/designed” to the exact length of the catheter, so as to not go beyond the tip, or enter into the bladder.

In use, the catheter bag is removed from the drainage lumen ending and/or the drainage tube is removed from the catheter. A user then inserts the catheter brush device with the blunt end first through the catheter opening to the end of the catheter. The device is always contained within the catheter to prevent perforation of the bladder. Once in position, the brush component is moved in a gentle, circular motion to clear any clotting and/or blockages. Once the clotting and/or blockages are cleared, the device is removed from the catheter. The drainage tubing and/or catheter bag is reattached and urine can flow freely through the catheter. If needed, the device can remain in the catheter and used to remove additional clots, blockages, etc. The process can be repeated as many times as necessary. As long as the device is not removed from the catheter, the brush component remains sterile, allowing for multiple blood clots and/or blockages to be removed. Accordingly, blood clots and blockages are removed without additional surgical incisions, which reduces trauma to the patient.

In yet another embodiment, the catheter brush device is manufactured from heat-sealable plastic or polymers, such as polypropylene or acrylonitrile-butadiene-styrene (ABS), silicon, etc.

In yet another embodiment, the catheter brush device is manufactured from a material that is water resistant.

In yet another embodiment, the catheter brush device comprises a plurality of indicia.

In another embodiment, the catheter brush device is packaged as a sterile kit. Specifically, a tray would be used to hold the components, such as multiple sizes of the catheter brush device, sterile gloves, mask, syringe, disinfectant wipes, etc. Typically, the catheter brush device comprises flexible material for the tubing and would be sterile packed in plastic.

In yet another embodiment, a method of removing blood clots from a Foley catheter is described. The method includes the steps of providing a catheter brush device comprising a brush with a blunt end with soft bristles and a loop at the other end which acts as a handle. The method also comprises removing the drainage tube from the Foley catheter. Further, the method comprises inserting the brush device through the opening to the tip of the catheter. The method also comprises moving the brush device in a gentle, circular motion to clear any clotting and blockages from the catheter. Finally, removing the brush device and reattaching the drainage tube, such that urine can flow freely through the catheter.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the catheter brush device of the present invention shown as a kit in accordance with the disclosed architecture;

FIG. 2 illustrates a perspective view of one potential embodiment of the catheter brush device of the present invention with the three-way irrigation channel in accordance with the disclosed architecture;

FIG. 3 illustrates a perspective view of one potential embodiment of the catheter brush device of the present invention wherein the device is inserted to access the blood clot in accordance with the disclosed architecture;

FIG. 4 illustrates a perspective view of one potential embodiment of the catheter brush device of the present invention wherein the blood clot is removed in accordance with the disclosed architecture;

FIG. 5 illustrates a perspective view of one potential embodiment of the catheter brush device of the present invention wherein the blunt tip and soft bristles are shown in accordance with the disclosed architecture; and

FIG. 6 illustrates a flowchart showing the method of removing blood clots from a Foley catheter using one potential embodiment of the catheter brush device of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a catheter brush device that provides users with a modified brush capable of removing blood clots and blockages from a Foley catheter during post-operative care. There is also a long-felt need in the art for a catheter brush device that features a blunt end brush with a loop end for holding, which allows users to insert the sterile brush into the catheter to secure and remove the clot. Further, there is a long-felt need in the art for a catheter brush device that allows the process of clot removal to be repeated, if sterility is maintained, to safely remove all clots without causing additional pain and trauma to a patient. Moreover, there is a long-felt need in the art for a device that maintains optimal flow for urine through the catheter when the drainage tubing is reconnected. Further, there is a long-felt need in the art for a catheter brush device that offers a more effective method of removing blood clots and debris from the bladder without requiring additional surgery. Finally, there is a long-felt need in the art for a catheter brush device that reduces trauma for a patient.

The present invention, in one exemplary embodiment, is a novel catheter brush device. The device is designed to offer a more effective method of removing blood clots and debris from the bladder without requiring additional surgery. The device comprises a brush with a blunt end with soft bristles, as well as a loop end that acts as a handle. Users remove the drainage tube from the catheter and insert the brush through the opening to the tip of the catheter. The brush is then moved in a gentle, circular motion to clear any clotting and blockages from the catheter. The brush is then removed, and the drainage tube reattached, such that urine can flow freely through the catheter. The present invention also includes a novel method of removing blood clots from a Foley catheter. The method includes the steps of providing a catheter brush device comprising a brush with a blunt end with soft bristles and a loop at the other end which acts as a handle. The method also comprises removing the drainage tube from the Foley catheter. Further, the method comprises inserting the brush device through the opening to the tip of the catheter. The method also comprises moving the brush device in a gentle, circular motion to clear any clotting and blockages from the catheter. Finally, removing the brush device and reattaching the drainage tube, such that urine can flow freely through the catheter.

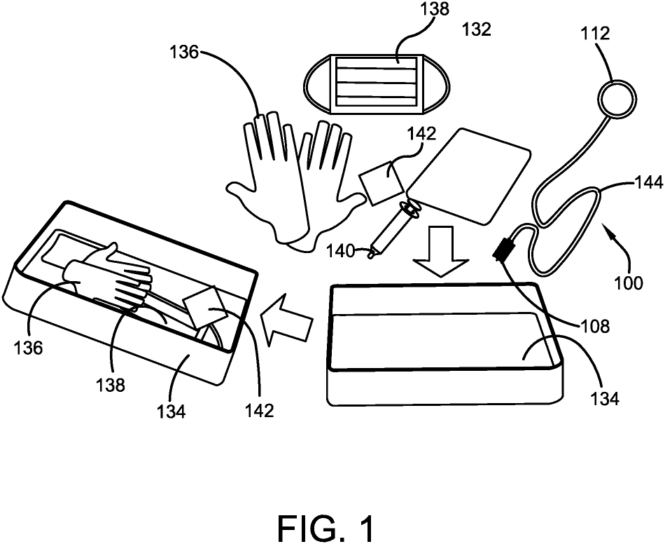

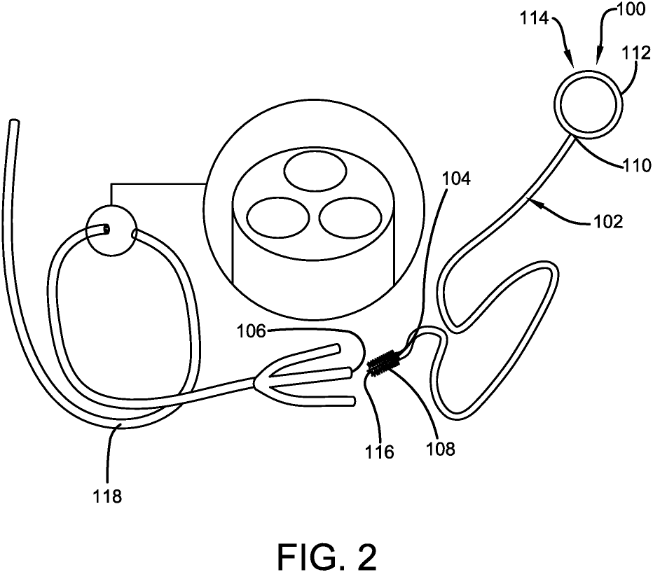

Referring initially to the drawings, FIGS. 1-2 illustrate a perspective view of one embodiment of the catheter brush device 100 of the present invention. In the present embodiment, the catheter brush device 100 is an improved catheter brush device that removes blood clots and debris from a Foley catheter or other suitable catheter as is known in the art. The device 100 is especially designed to maintain optimal flow for urine through the catheter for any suitable user as is known in the art, such as nurses, doctors. More specifically, the device 100 comprises a brush component 102 with a first end 104 comprising a blunt end 106 with a plurality of soft bristles 108 and a second end 110 comprising a wire loop 112 that acts as a handle 114. Typically, users insert the brush device 100 with the blunt end 106 leading through the opening 116 of the catheter 118. The brush device 100 is then moved in a gentle, circular motion to clear any clotting and blockages from the catheter 118. The brush device 100 is then removed, such that urine can flow freely through the catheter 118.

The catheter brush device 100 is typically used with a standard Foley catheter 118 as is known in the art but can be utilized with any suitable catheter as is known in the art, depending on the needs and/or wants of a user. Generally, the Foley catheter 118 comprises a proximal end 122 and a distal end 124. The proximal end 122 comprises side drainage openings 126, wherein as the distal end 124 of the catheter 118 preferably has three lumen endings 128. Typically, the largest diameter lumen ending is preferably the drainage lumen 130 extending from the side drainage openings 126 to the drainage lumen ending 130. This drainage lumen ending 130 is preferably used to drain urine from the bladder 120. The drainage lumen ending 130 preferably has a fitting (not shown) to connect the lumen to a catheter bag (not shown). Furthermore, the catheter lumens 128 are preferably made of silicon, or any other suitable flexible material as is known in the art.

In one embodiment, the catheter brush device 100 is packaged as a sterile kit 132, as shown in FIG. 1. Specifically, a tray 134 or other suitable device would be used to hold the components, such as a standard size of the catheter brush device 100, sterile gloves 136, mask 138, syringe 140, disinfectant wipes 142, etc., or any other suitable components as is known in the art, depending on the needs and/or wants of a user. Typically, the catheter brush device 100 comprises flexible material for the tubing 144 and would be sterile packed in plastic.

Generally, the catheter brush device 100 comprises a brush component 102 with a first end 104 comprising a blunt end 106 with a plurality of soft bristles 108. The blunt end 106 can be any suitable shape as is known in the art, as long as the end does not comprise any sharp edges to damage the catheter 118 during insertion. Typically, the blunt end 106 is a rounded end. The plurality of soft bristles 108 are then secured to the blunt end 106 via any suitable securing means as is known in the art, such as glue, adhesive, etc. Any suitable number of bristles 108 can be utilized as is known in the art, depending on the needs and/or wants of a user. Typically, bristles 108 are secured all around the blunt end 106 and down the sides forming a tube-like shape of bristles 108 at the blunt end 106. In one embodiment, more bristles 108 can be utilized on the catheter brush device 100 for removing large blood clots and clogs within the catheter 118, and less bristles 108 can be utilized on the catheter brush device 100 for removing smaller blood clots and clogs within the catheter 118. Typically, any size catheter brush device 100 with any number of bristles 108 can be utilized as is known in the art, depending on the needs and/or wants of a user, as long as they meet the specific diameter of the foley lumen.

Furthermore, the brush component 102 of the catheter brush device 100 comprises a second end 110 comprising a wire loop 112 that acts as a handle 114. Typically, the second end 110 can comprise any suitable shape as is known in the art, such as a circle, square, rectangle, etc., as long as a user can easily grasp the second end 110 like a handle 114 when using the device 100. In one embodiment, the second end 110 can be straight and not have a loop 112 or other shape. In this embodiment, the user would merely grasp the straight second end 110 to manipulate the device 100.

Additionally, the brush component 102 comprises a middle section 146 of flexible tubing 144. The middle section 146 can be any suitable size as is known in the art, depending on the size of the catheter 118 in use or based on the needs and/or wants of a user. The middle section 146 of flexible tubing 144 allows the device 100 to be threaded up through a catheter 118 for use.

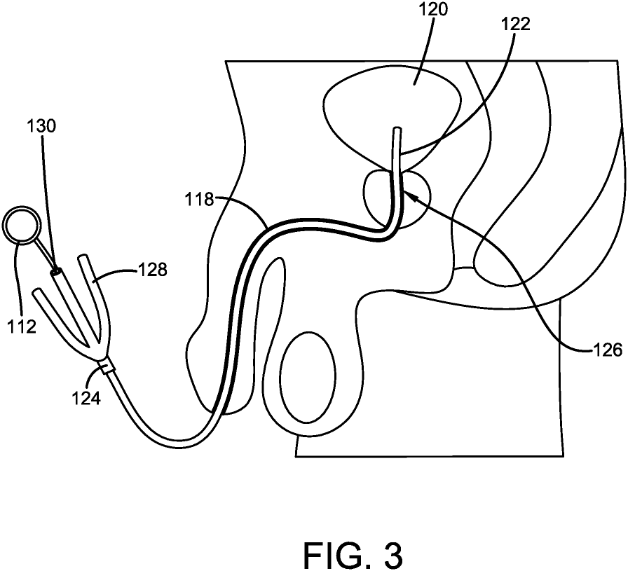

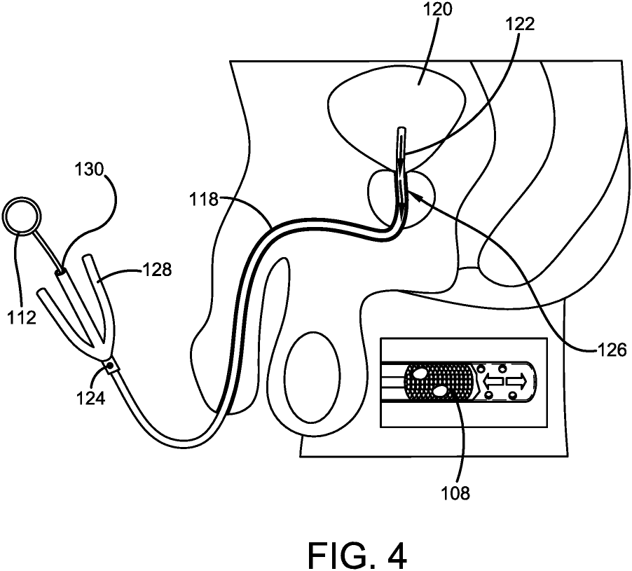

As shown in FIGS. 3-4, in use, the catheter bag (not shown) is removed from the drainage lumen ending 130 and/or the drainage tube 130 is removed from the catheter 118. A user then inserts the catheter brush device 100 with the blunt end 106 first through the catheter opening 116 to the end of the catheter 118. The device 100 is always contained within the catheter 118 to prevent perforation of the bladder 120. Once in position, the brush component 102 is moved in a gentle, circular motion to clear any clotting and/or blockages. Once the clotting and/or blockages are cleared, the device 100 is removed from the catheter 118. The drainage tubing 130 and/or catheter bag is re-attached and urine can flow freely through the catheter 118. If needed, the device 100 can remain in the catheter 118 and used to remove additional clots, blockages, etc. The process can be repeated as many times as necessary. As long as the device 100 has sterility maintained, the brush component 102 remains sterile, allowing for multiple blood clots and/or blockages to be removed. Accordingly, blood clots and blockages are removed without additional surgical incisions, which reduces trauma for the patient.

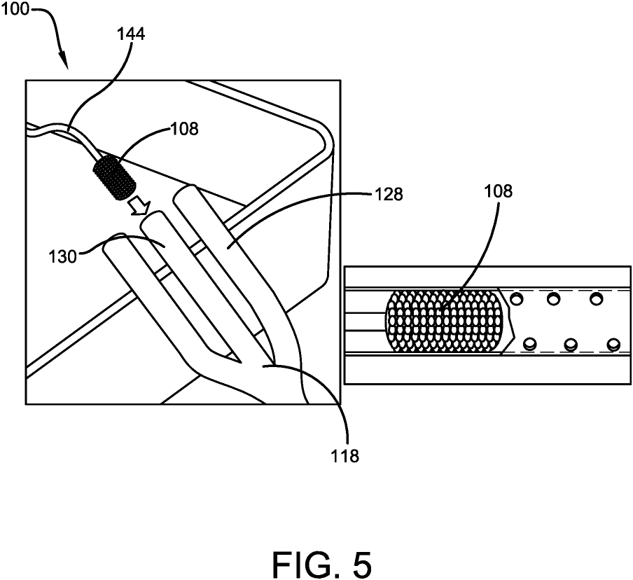

As shown in FIG. 5, the catheter brush device 100 is manufactured from heat-sealable plastic or polymers, such as polypropylene or acrylonitrile-butadiene-styrene (ABS), silicon, or any other suitable material as is known in the art, such as, but not limited to, polyethylene, thermoplastics, polyethylene terephthalate, polyvinyl chloride, polystyrene, etc. Generally, the catheter brush device 100 is also manufactured from a material that is water resistant or water permeable or comprises a coating that is water resistant or water permeable. The catheter brush device 100 is also reusable provided that it is properly sterilized between uses.

In another embodiment, the catheter brush device 100 comprises a plurality of indicia 500. The brush component 102 of the device 100 may include advertising, a trademark, or other letters, designs, or characters, printed, painted, stamped, or integrated into the brush component 102, or any other indicia 500 as is known in the art. Specifically, any suitable indicia 500 as is known in the art can be included, such as, but not limited to, patterns, logos, emblems, images, symbols, designs, letters, words, characters, animals, advertisements, brands, etc., that may or may not be catheter or health/brand related.

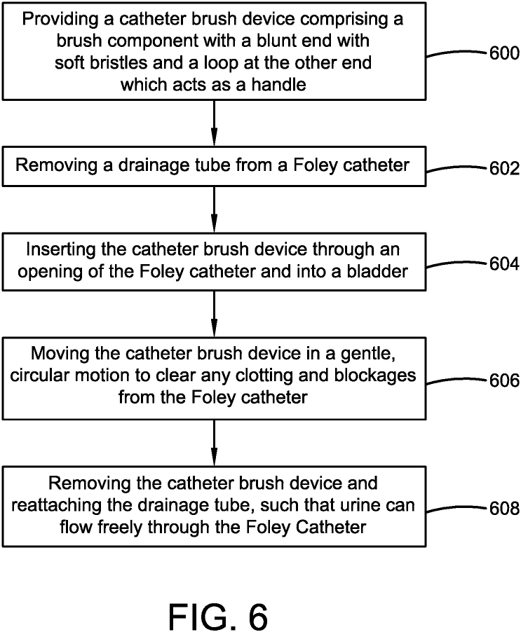

FIG. 6 illustrates a flowchart of the method of removing blood clots from a Foley catheter. The method includes the steps of at 600, providing a catheter brush device comprising a brush component with a blunt end with soft bristles and a loop at the other end which acts as a handle. The method also comprises at 602, removing the drainage tube from the Foley catheter. Further, the method comprises at 604, inserting the brush device through the opening to the tip of the catheter. The method also comprises at 606, moving the brush device in a gentle, circular motion to clear any clotting and blockages from the catheter, wherein the process can be repeated to remove additional clots and blockages, once the debris/clots are removed from the brush. Finally, at 608, removing the brush device and reattaching the drainage tube, such that urine can flow freely through the catheter.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “catheter brush device”, “brush device”, and “device” are interchangeable and refer to the catheter brush device 100 of the present invention.

Notwithstanding the foregoing, the catheter brush device 100 of the present invention can be of any suitable size and configuration as is known in the art without affecting the overall concept of the invention, provided that it accomplishes the above-stated objectives. One of ordinary skill in the art will appreciate that the catheter brush device 100 as shown in FIGS. 1-6 are for illustrative purposes only, and that many other sizes and shapes of the catheter brush device 100 are well within the scope of the present disclosure. Although the dimensions of the catheter brush device 100 are important design parameters for user convenience, the catheter brush device 100 may be of any size that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A catheter brush device that removes blood clots and debris from a Foley catheter, the catheter brush device comprising:
a brush component comprised of a first end, a second end and a middle section;
wherein the first end comprises a plurality of bristles;
wherein the middle section comprises a tubing; and
further wherein the brush component is inserted into the Foley catheter to remove blood clots and maintain urine flow through the Foley catheter.

2. The catheter brush device of claim 1, wherein the first end comprises a blunt end.

3. The catheter brush device of claim 2, wherein the second end comprises a wire loop that functions as a handle.

4. The catheter brush device of claim 3, wherein the blunt end is inserted through an opening of the Foley catheter and into a bladder.

5. The catheter brush device of claim 4, wherein once through the opening of the Foley catheter, the blunt end is moved in a gentle, circular motion to clear any clotting and blockages.

6. The catheter brush device of claim 1, wherein the Foley catheter comprises a proximal end and a distal end, with the proximal end comprising side drainage openings and the distal end comprising three lumen endings.

7. The catheter brush device of claim 5, wherein the catheter brush device is packaged as a sterile kit.

8. The catheter brush device of claim 7, wherein the sterile kit comprises a tray comprised of a pair of sterile gloves, a mask, a syringe, a plurality of disinfectant wipes, and at least one catheter brush device.

9. The catheter brush device of claim 1, wherein the tubing is comprised of a flexible material, such that the catheter brush device can be threaded through the Foley catheter.

10. The catheter brush device of claim 2, wherein the plurality of bristles are secured around the blunt end.

11. The catheter brush device of claim 1 further comprising a plurality of indicia.

12. The catheter brush device of claim 1, wherein the catheter brush device is manufactured from a polypropylene, an acrylonitrile-butadiene-styrene (ABS), a silicon, a polyethylene, a polyethylene terephthalate, a polyvinyl chloride or a polystyrene.

13. The catheter brush device of claim 12, wherein the catheter brush device is also manufactured from a material that is water resistant or water permeable.

14. A catheter brush device that removes blood clots and debris from a Foley catheter, the catheter brush device comprising:
a brush component comprised of a first end, a second end and a middle section;
wherein the first end comprises a blunt end having a plurality of bristles;
wherein the second end comprises a wire loop that acts as a handle;
wherein the middle section comprises a flexible tubing; and
wherein the brush component is inserted into a Foley catheter to remove blood clots and maintain urine flow through the Foley catheter;
wherein once through the Foley catheter, the blunt end is moved in a gentle, circular motion to clear any clotting and blockages; and
further wherein process can be repeated to remove additional clots and blockages, as long as the catheter brush device is not removed from the Foley catheter.

15. The catheter brush device of claim 14, wherein the catheter brush device is packaged as a sterile kit.

16. The catheter brush device of claim 15, wherein the sterile kit comprises a tray comprised of a pair of sterile gloves, a mask, a syringe, a plurality of disinfectant wipes, and at least one catheter brush device.

17. The catheter brush device of claim 14 further comprising a plurality of indicia.

18. The catheter brush device of claim 14, wherein the plurality of bristles are secured around the blunt end.

19. A method of removing blood clots from a Foley catheter comprising the steps of:
providing a catheter brush device comprising a brush component with a blunt end with soft bristles and a loop at other end which acts as a handle;
removing a drainage tube from a Foley catheter;
inserting the catheter brush device through an opening of the Foley catheter and into a bladder;
moving the catheter brush device in a gentle, circular motion to clear any clotting and blockages from the Foley catheter; and
removing the catheter brush device and reattaching the drainage tube, such that urine can flow freely through the Foley catheter.

20. The method of claim 19, wherein the process can be repeated to remove additional clots and blockages, as long as the catheter brush device is not removed from the Foley catheter.
